(19) United States
(12) Patent Application Publication
(10) Pub. No.: US 2017/0156978 A1
(43) Pub. Date: Jun. 8, 2017

(54) CARDIOPULMONARY RESUSCITATION SYSTEM, CARDIOPULMONARY RESUSCITATION DEVICE, AND ARTIFICIAL RESPIRATOR

(71) Applicant: KOHKEN MEDICAL CO., LTD., Tokyo (JP)

(72) Inventors: Eiichi MATSUI, Tokyo (JP); Yukio SEINO, Tokyo (JP); Mitsuo MATSUI, Tokyo (JP); Hitoshi TAZAKI, Tokyo (JP)

(21) Appl. No.: 15/325,694

(22) PCT Filed: Jul. 28, 2015
(86) PCT No.: PCT/JP2015/071402

§ 371 Date: Jan. 11, 2017

(30) Foreign Application Priority Data
Jul. 29, 2014 (JP) 2014-154294

(51) Int. Cl. A61H 31/00; A61M 16/20; A61M 16/00

(52) U.S. Cl. CPC A61H 31/006; A61M 16/0063; A61M 16/0003; A61M 16/202; A61M 2016/0027; A61M 2202/0208; A61H 2201/5071

(57) ABSTRACT

An object of the present invention is to provide a cardiopulmonary resuscitation system in which a cardiopulmonary resuscitation device is made compact and which is capable of performing blowing of breathable gas into a patient at more appropriate timing. A cardiopulmonary resuscitation system (1) according to the present invention is equipped with a cardiopulmonary resuscitation device (100), an artificial respirator (200), and a signal transmission unit (300) which transmits an external signal to an external signal input unit (140) from an external signal output unit (240), and has a local mode in which a first gas blowing unit (110) and a chest compression unit (120) are operable and a second gas blowing unit (210) is stopped, and a remote mode in which the chest compression unit and the second gas blowing unit are operable and the first gas blowing unit is stopped, and in the remote mode, the cardiopulmonary resuscitation device and the artificial respirator are capable of transmitting an external signal by the signal transmission unit, and a second control unit (230) controls the chest compression unit.

### TECHNICAL FIELD

The present invention relates to a cardiopulmonary resuscitation system, and a cardiopulmonary resuscitation device and an artificial respirator used therefor.

### BACKGROUND ART

As cardiopulmonary resuscitation, a method has been known in which chest compression by hand and artificial respiration by a mouth-to-mouth method are performed in combination. However, it is difficult to perform stable and high-quality cardiopulmonary resuscitation by hand. Therefore, a cardiopulmonary resuscitation device in which chest compression and artificial respiration are automated has been proposed. For example, the applicant has proposed an automatic cardiopulmonary resuscitation device which performs cardiac massage by repeatedly applying an impact at adjusted constant time intervals, and ventilates and supplies breathable gas at adjusted time and for an adjusted period (for example, see Patent Literature 1).

### CITATION LIST

### Patent Literature

Patent Literature 1: JP 2000-84028 A

### SUMMARY OF INVENTION

### Technical Problem

In conventional cardiopulmonary resuscitation devices, “pushing” for forcibly injecting breathable gas into the lungs of a patient is performed during artificial respiration. In addition, at time of chest compression, passive ventilation occurs in which air is taken into the lungs of a patient when the compressed chest wall of the patient recoils. In order to further improve a resuscitation rate, it is more preferable to perform “active ventilation” in which gas adjusted as breathable gas is blown into the patient at timing when the compressed chest wall of the patient recoils at the time of chest compression, and thereby the breathable gas is taken into the lungs of the patient, than to perform the passive ventilation. However, there has been a problem that although it is important to make a cardiopulmonary resuscitation device compact in consideration of use thereof in the emergency medical care field, the cardiopulmonary resuscitation device has to be increased in size in order to more securely perform the active ventilation with the cardiopulmonary resuscitation device. Therefore, not the active ventilation, but just the passive ventilation has been performed in conventional cardiopulmonary resuscitation devices.

An object of the present invention is to provide a cardiopulmonary resuscitation system in which a cardiopulmonary resuscitation device is made compact and which is capable of performing blowing of breathable gas into a patient at more appropriate timing. Another object of the present invention is to provide a cardiopulmonary resuscitation device and an artificial respirator suitable for the cardiopulmonary resuscitation system.

### Solution to Problem

A cardiopulmonary resuscitation system according to the present invention is equipped with: a cardiopulmonary resuscitation device that has a first gas blowing unit that blows breathable gas into a patient, a chest compression unit that compresses the chest of the patient, a first control unit that controls the first gas blowing unit and the chest compression unit, and an external signal input unit that inputs an external signal including a remote control signal instructing the chest compression unit to execute the chest compression; an artificial respirator that has a second gas blowing unit that blows breathable gas into the patient, a second control unit that controls the second gas blowing unit and generates the external signal, an external signal output unit that outputs the external signal generated by the second control unit to outside, and an airway internal pressure sensor that detects pressure in the airway of the patient; and a signal transmission unit that transmits the external signal to the external signal input unit from the external signal output unit, and has: a local mode in which the first gas blowing unit and the chest compression unit are operable and the second gas blowing unit is stopped; and a remote mode in which the chest compression unit and the second gas blowing unit are operable and the first gas blowing unit is stopped, in which in the remote mode, the cardiopulmonary resuscitation device and the artificial respirator are capable of transmitting the external signal by the signal transmission unit, and the second control unit controls the chest compression unit.

In the cardiopulmonary resuscitation system according to the present invention, preferably, the cardiopulmonary resuscitation device has a mode-switching button for performing switching between the local mode and the remote mode, or the external signal includes a mode-switching signal for performing switching between the local mode and the remote mode. Switching from the local mode to the remote mode can be performed easily and quickly.

Regarding the cardiopulmonary resuscitation system according to the present invention, in the remote mode, the second gas blowing unit preferably executes blowing of the breathable gas at timing when the airway internal pressure sensor detects negative pressure. Active ventilation can be executed.

A cardiopulmonary resuscitation device according to the present invention has: a first gas blowing unit that blows breathable gas into a patient; a chest compression unit that compresses the chest of the patient; a first control unit that controls the first gas blowing unit and the chest compression unit; and an external signal input unit that inputs an external signal including a remote control signal instructing the chest compression unit to execute the chest compression.

An artificial respirator according to the present invention has: a second gas blowing unit that blows breathable gas into a patient; a second control unit that controls the second gas blowing unit and generates an external signal including a remote control signal to a cardiopulmonary resuscitation device; an external signal output unit that outputs the external signal generated by the second control unit to outside; and an airway internal pressure sensor that detects pressure in the airway of the patient.

### Advantageous Effects of Invention

The present invention can provide a cardiopulmonary resuscitation system in which a cardiopulmonary resuscitation device is made compact and which is capable of performing blowing of breathable gas into a patient at more appropriate timing. In addition, the present invention can provide a cardiopulmonary resuscitation device and an artificial respirator suitable for the cardiopulmonary resuscitation system.

### BRIEF DESCRIPTION OF DRAWINGS

FIG. 1 is an example of a block diagram of a cardiopulmonary resuscitation device according to an embodiment.

FIG. 2 is an example of a block diagram of an artificial respirator according to the embodiment.

FIG. 3 is an example of a conceptual diagram of a cardiopulmonary resuscitation system according to the embodiment.

FIG. 4 is a drawing for explaining use of the cardiopulmonary resuscitation system according to the embodiment.

FIG. 5 is a perspective view exemplifying the cardiopulmonary resuscitation device according to the embodiment.

### DESCRIPTION OF EMBODIMENTS

Next, the present invention will be described in detail by describing embodiments, but the present invention is not construed as being limited to description thereof. As long as an effect of the present invention is exhibited, the embodiments may be modified variously.

(Cardiopulmonary Resuscitation Device)

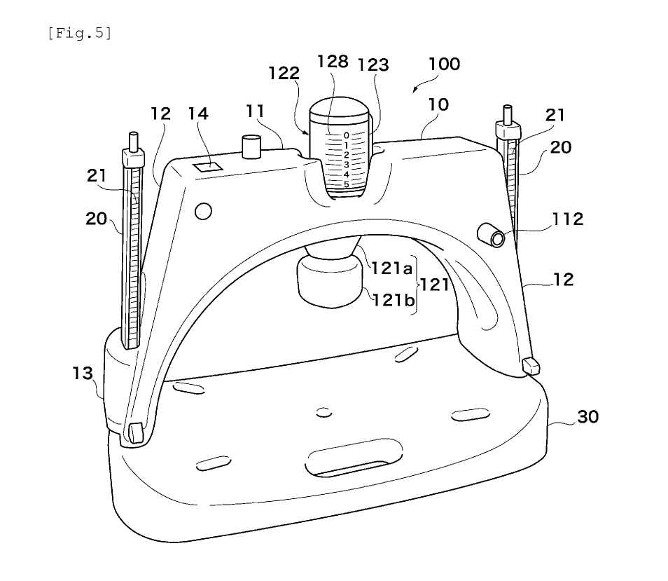

FIG. 5 is a perspective view exemplifying a cardiopulmonary resuscitation device according to the embodiment. As illustrated in FIG. 5, the cardiopulmonary resuscitation device 100 is equipped with an arch portion 10, vertical rods 20, and a back plate 30.

The arch portion 10 has a top surface portion 11 and right and left side surface portions 12, and is disposed astride an upper portion of the chest of a patient. The arch portion 10 has an impact hammer 121 projecting downward from the top surface portion 11 and supported by the top surface portion 11 in a vertically movable manner, and an elevating means 122 which vertically reciprocates the impact hammer 121. The impact hammer 121 has an impact hammer rod 121a coupled to the elevating means 122 and an impact head pad 121b attached to a lower end portion of the impact hammer rod 121a and applied to the chest of the patient. In the impact hammer rod 121a and the impact head pad 121b, it is preferable to provide an angle adjusting function for the impact head pad 121b at a tip end portion of the impact hammer rod 121a such that a pad surface of the impact head pad 121b always in parallel with the sternum of the patient. In other words, it is preferable for the impact hammer 121 to be a structure in which the angle of the impact head pad 121b is freely arranged at the tip end portion of the impact hammer rod 121a. The impact head pad 121b consists of, for example, a soft elastic body material such as a silicone resin. In addition, a portion of the impact head pad 121b applied to the chest of the patient preferably has a diameter of 5 cm to 8 cm.

In addition, although there is no particular limitation, hardness of the impact head pad 121b is preferably soft. For example, when the impact head pad 121b is made of silicone rubber, the hardness thereof is preferably about 20. Although there may be a risk of increasing the frequency of costal cartilage fractures by making the diameter of the impact head pad 121b larger, the frequency of bone fractures can be reduced even when making the diameter of the impact head pad 121b larger, by softening the hardness of the impact head pad 121b to absorb a load on the costal cartilage. In addition, by making the impact head pad 121b as a structure in which the angle of the impact head pad 121b is freely arranged at the tip end portion of the impact hammer rod 121a such that the pad surface always in parallel with the sternum of the patient, it is possible to prevent sternum fractures (costal cartilage fractures) caused by shift in a position where a compression pad is pressed.

Furthermore, by providing the angle adjusting function for the impact head pad 121b at the tip end portion of the impact hammer rod 121a, a structure is obtained in which the impact head pad 121b flexibly moves, which makes it possible to perform chest compression in a state where the angle of the pad surface of the impact head pad 121b is always in parallel with the sternum. Consequently, it is possible to disperse a condition of a pressing load on convexity and concavity of the sternum to a plane from a point, and therefore to contribute prevention of sternum fractures.

The impact hammer 121 preferably has, in a central portion thereof, a laser irradiation unit (not illustrated) which performs irradiation with laser light along a vertical reciprocating direction of the impact hammer 121. A compression position can be more easily confirmed by irradiating a patient with laser light. In addition, a configuration may be adopted in which the chest thickness of a patient is measured by irradiating the chest of the patient with laser light, and receiving reflection thereof. Preferably, the impact head pad 121b has, in a central portion thereof, a through hole (not illustrated) provided so as to penetrate the impact head pad 121b along the vertical reciprocating direction of the impact hammer 121, and the laser irradiation unit is disposed in the through hole of the impact head pad 121b. The laser irradiation unit is, for example, a green laser pointer.

An operation to align a compression position of the heart of the patient and the position of the impact head pad 121b is preferably performed manually as follows. By visual confirmation by a laser point from the central portion of the impact hammer 121, with which the patient is irradiated, a position of the laser point and an optimal point of a compression site of the patient are made to coincide with each other. In a case where the position of the laser point shifts from the optimal point of the compression site of the patient at that time, the cardiopulmonary resuscitation device 100 is moved such that the laser point is applied to the optimal point of the compression site of the patient, while slightly lifting the back of the patient and visually confirming the laser point with which the chest of the patient is irradiated. By this operation, the impact head pad 121b can be more securely applied to the optimal point of the compression site of the patient. Even if the optimal point of the compression site of the patient is somewhat shifted up and down and right and left by the sway of a vehicle while carrying the patient, inclination of a stretcher while carrying the patient, or the like, by providing the angle adjusting function for the impact head pad 121b at the tip end portion of the impact hammer rod 121a, compression is performed in a state where the angle of the pad surface of the impact head pad 121b is always in parallel with the sternum of the patient, which makes it possible to disperse a load of the pad to a plane from a point, and to prevent chest compression.

The vertical rods 20 are provided at right and left in pairs, and fixed to fixing units 13 provided in lower ends of right and left side surface portions 12 of the arch portion, respectively. Each of the vertical rod 20 is engaged, for example, with a ratchet of the fixing unit 13 and supports the arch portion 10 in a vertically movable manner. In each of the vertical rod 20, a scale 21 is preferably indicated as illustrated in FIG. 5. Preferably, while keeping a state where the arch portion 10 is set on the patient, the arch portion 10 is pushed down toward the chest of the patient, and when the impact head pad 121b contacts the chest of the patient, the scale is read and recorded as the chest thickness of the patient. A compression depth of the impact hammer 121 can be set based on the read chest thickness. By the configuration described above, finer adjustment of the compression depth suitable for each one of patients can be performed.

In the cardiopulmonary resuscitation device 100 according to the embodiment, when the arch portion 10 is provided at an upper portion of a patient, the arch portion 10 can be lowered by the vertical rods 20 in accordance with the chest thickness of the patient. By that operation, the center of gravity of the cardiopulmonary resuscitation device 100 can be lowered, and therefore, the chest compression can be continued stably even when the patient with the cardiopulmonary resuscitation device 100 mounted thereon is carried on a stretcher and the stretcher is inclined, for example, on the stairs. In addition, it is possible to prevent fractures of the sternum or the rib caused by shift of the compression site.

The back plate 30 is a plate which supports an undersurface of the chest of a patient. The back plate 30 is detachably fixed to the arch portion 10, for example, by engaging an engaging portion (not illustrated) such as a groove or a hole provided in the back plate 30 with a protrusion (not illustrated) provided at a lower end of the vertical rod 20.

The cardiopulmonary resuscitation device 100 preferably has a display unit 14. The display unit 14 displays, for example, the chest thickness of a patient measured by irradiation with laser light, the compression depth, or a load during the chest compression of the patient. The display unit 14 preferably displays at least both of the compression depth and the load during the chest compression of the patient. When the depth or the load during the compression is too large, fractures of the sternum or the rib may be caused. The fractures of the sternum or the rib greatly affect social rehabilitation, and in addition thereto, when the patient fracture the sternum or the rib, the impact head pad 121b cannot return to the default position. In that case, there may be a risk of preventing blood circulation since the blood is difficult to return to the heart, and therefore, prevention of fractures of the sternum or the rib is desired. In addition, regarding the compression depth and the load, although it is recommended according to guidelines to push to 5 cm with a load of 50 kg, the hardnesses of the sternum and the rib are different with each person, and adjustment is required for each one of patients. By displaying and confirming the compression depth and the load during the compression on the display unit 14, the hardnesses of the sternum and the rib of the patient can be found. As a result, it is possible to prevent the fractures of the sternum or the rib, and to further increase the safety.

The display unit 14 may display all display items on one display unit, or a separate display unit may be provided for each display item. In addition, although in FIG. 5, an example is illustrated in which the display unit 14 is provided on the arch portion 10, the present invention is not limited thereto, and the display unit 14 may be provided, for example, on the back plate 30.

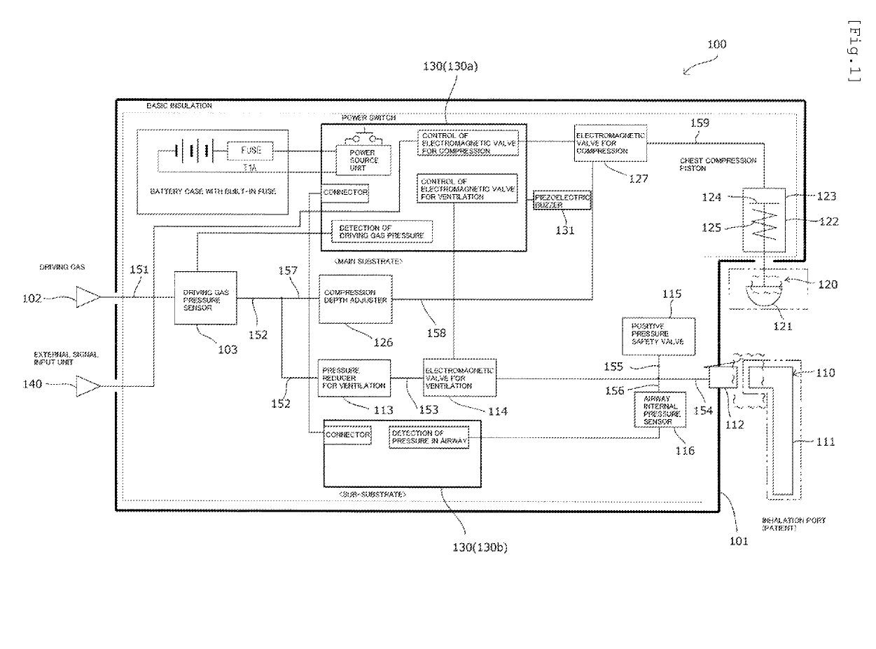

FIG. 1 is an example of a block diagram of the cardiopulmonary resuscitation device according to the embodiment. The cardiopulmonary resuscitation device 100 will be described with reference to FIG. 1. The cardiopulmonary resuscitation device 100 according to the embodiment has a first gas blowing unit 110 which blows breathable gas into a patient, a chest compression unit 120 which compresses the chest of the patient, a first control unit 130 which controls the first gas blowing unit 110 and the chest compression unit 120, and an external signal input unit 140 which inputs an external signal including a remote control signal instructing the chest compression unit 120 to execute the chest compression.

The cardiopulmonary resuscitation device 100 has a housing 101 which accommodates the first control unit 130, a driving system of the first gas blowing unit 110, a driving system of the chest compression unit 120, and the like. There is no particular limitation for a position where the housing 101 is provided, and for example, the housing 101 may be provided as apart of the arch portion 10 (illustrated in FIG. 5) disposed astride the upper portion of the chest of the patient, may be externally attached to the arch portion 10 (illustrated in FIG. 5), may be provided as a part of the back plate 30 (illustrated in FIG. 5) which supports the undersurface of the chest of the patient, or may be externally attached to the back plate 30 (illustrated in FIG. 5).

The first gas blowing unit 110 has a hose 111 for blowing breathable gas into the patient. One end portion of the hose 111 is coupled to a hose outlet 112 provided in the housing 101 of the cardiopulmonary resuscitation device 100. Another end portion of the hose 111 is coupled to a mask (not illustrated) to be attached to the patient or a tube for tracheal intubation (not illustrated).

The driving system of the first gas blowing unit 110 includes, for example, a driving gas supply source 102, a driving gas pressure sensor 103, a pressure reducer for ventilation 113, an electromagnetic valve for ventilation 114, a positive pressure safety valve 115, an airway internal pressure sensor 116, and pipes 151 to 156 for connecting those described above. The pipe 151 connected to the driving gas supply source 102 is connected to the driving gas pressure sensor 103. The pipe 152 connected to the driving gas pressure sensor 103 is connected to the pressure reducer for ventilation 113. The pipe 153 connected to the pressure reducer for ventilation 113 is connected to the electromagnetic valve for ventilation 114. The pipe 154 connected to the electromagnetic valve for ventilation 114 is connected to the hose outlet 112. The positive pressure safety valve 115 is connected to the pipe 154 with the pipe 155. The airway internal pressure sensor 116 is connected to the pipe 154 with the pipe 156.

The driving gas supply source 102 is, for example, a gas cylinder or an air tank. The driving gas supply source 102 is preferably a portable gas cylinder in terms of excellent portability. The types of the driving gas are, for example, pure oxygen, oxygen-enriched air or air. Pressure of the driving gas supplied from the driving gas supply source 102 is reduced to predetermined pressure by a regulator (not illustrated) and the driving gas is sent to the driving gas pressure sensor 103. The predetermined pressure is preferably pressure suitable for driving the chest compression unit 120, and is preferably adjusted, for example, to 0.35 to 0.45 MPa. The driving gas pressure sensor 103 detects the pressure of gas supplied from the driving gas supply source 102, and outputs a pressure signal to the first control unit 130. The first control unit 130 sounds a piezoelectric buzzer when the pressure is higher or lower than a set value based on the input pressure signal. The driving gas which has passed through the driving gas pressure sensor 103 is sent to the pressure reducer for ventilation 113. The pressure reducer for ventilation 113 reduces the pressure of the driving gas to pressure suitable for breathing to generate breathable gas. The breathable gas is sent to the electromagnetic valve for ventilation 114. The opening and closing of the electromagnetic valve for ventilation 114 is controlled by the first control unit 130, and thereby ON/OFF control of gas discharged from the hose outlet 112 is performed. The positive pressure safety valve 115 is a relief valve which is opened when pressure in the pipe 154 between the electromagnetic valve for ventilation 114 and the hose outlet 112 reaches abnormal pressure (for example, 70 hPa or greater), such as a case where the airway of the patient is obstructed. The airway internal pressure sensor 116 detects pressure in the pipe 154 between the electromagnetic valve for ventilation 114 and the hose outlet 112. The pressure in the pipe 154 is regarded as airway internal pressure of the patient. It is sufficient that the airway internal pressure sensor 116 can detect at least positive pressure. The airway internal pressure sensor 116 outputs a pressure signal to the first control unit 130. The first control unit 130 sounds the piezoelectric buzzer when the pressure is higher than a set value based on the input pressure signal. In addition, the first control unit 130 outputs, to the electromagnetic valve for ventilation 114, a signal which causes the electromagnetic valve for ventilation 114 to close when the pressure detected by the airway internal pressure sensor 116 reaches at least a predetermined pressure value (for example, greater than 40 hPa). As a result, it is possible to prevent high-pressure gas from being injected into the patient.

The driving gas of the first gas blowing unit 110 is preferably pure oxygen. By blowing pure oxygen into the patient at recoiling, blood circulation is maintained and more efficient oxygenation can be performed without increasing intrathoracic pressure. Since it is possible to blow about five times as much oxygen as that in a case of blowing air, it is effective to prevent necrosis of the brain and the organs to improve cerebral performance category (CPC) and overall performance category (OPC).

A ventilation amount of the breathable gas blown by the first gas blowing unit 110 is adjusted, for example, by controlling a diaphragm or a needle valve provided in the pressure reducer for ventilation 113. In addition, inhalation time of the breathable gas blown by the first gas blowing unit 110 is adjusted, for example, by controlling opening time of the electromagnetic valve for ventilation 114 by the first control unit 130. The ventilation amount of the breathable gas blown by the first gas blowing unit 110 is adjusted, for example, to 200 to 1200 ml for one time, and the inhalation time is adjusted stepwise, for example, to 1.0 seconds, 1.5 seconds, or 2.0 seconds. As a result, a flow rate of the breathable gas blown by the first gas blowing unit 110 can be adjusted in a range of, for example, from 12 to 36 litters/min. In the cardiopulmonary resuscitation system according to the embodiment, an artificial respirator can perform artificial respiration in which the flow rate and blowing timing of the breathable gas are more strictly adjusted, and therefore, the flow rate and the blowing timing of the breathable gas blown by the first gas blowing unit 110 may be adjusted by a relatively simple method, such as opening and closing of the diaphragm or the needle valve provided in the pressure reducer for ventilation, and an electromagnetic valve. As a result, it is possible to reduce the size and the weight of the cardiopulmonary resuscitation device 100.

The chest compression unit 120 has the impact hammer 121 which applies an impact on the chest of the patient, and the elevating unit 122 which vertically reciprocates the impact hammer 121. The elevating unit 122 has a cylinder 123. The cylinder 123 is in a container shape, and has a gas supply port (not illustrated) and a gas discharge port (not illustrated). In a space inside the cylinder 123, a piston 124 and a spring 125 which pushes back the piston 124 when discharging gas are disposed.

The driving system of the chest compression unit 120 includes, for example, a driving gas supply source 102, a driving gas pressure sensor 103, a compression depth adjuster 126, an electromagnetic valve for compression 127, and pipes 151, 152, 157 to 159 for connecting those described above. The pipe 151 connected to the driving gas supply source 102 is connected to the driving gas pressure sensor 103. The pipe 152 connected to the driving gas pressure sensor 103 is connected to the pipe 157 and the pipe 157 is connected to the compression depth adjuster 126. The pipe 158 connected to the compression depth adjuster 126 is connected to the electromagnetic valve for compression 127. The pipe 159 connected to the electromagnetic valve for compression 127 is connected to the elevating unit 122.

The driving gas supply source 102 and the driving gas pressure sensor 103 are preferably used also as the driving gas supply source 102 and the driving gas pressure sensor 103 of the driving system of the first gas blowing unit 110. The compression depth adjuster 126 adjusts a stroke width of vertical reciprocation of the elevating unit 122. Although the stroke width of vertical reciprocation is appropriately adjusted for each patient, it is recommended in the “2010 AHA Guidelines for CPR and ECC” (hereinafter also referred to as guidelines) that the stroke width of vertical reciprocation is 5 cm or more for adult. The electromagnetic valve for compression 127 is, for example, a three-way electromagnetic valve. Alternatively, separate valves may be used for a gas input valve and a gas discharge valve, as the electromagnetic valve for compression 127 instead of the three-way electromagnetic valve. Opening and closing of the electromagnetic valve for compression 127 is controlled by the first control unit 130. When a driving gas is supplied into the cylinder 123, the piston 124 is pushed down while resisting to repulsive force of the spring 125, and thereby the impact hammer 121 moves downward. When the driving gas is discharged from the cylinder 123, the spring 125 extends to push up the piston 124, and thereby the impact hammer 121 moves upward. By repeating the above operations, the impact hammer 121 vertically reciprocates.

Although a mode is indicated in FIG. 1 in which the driving gas supply source 102 and the driving gas pressure sensor 103 in the driving system of the chest compression unit 120 are used also as the driving gas supply source 102 and the driving gas pressure sensor 103 of the driving system of the first gas blowing unit 110, the driving gas supply source 102 and the driving gas pressure sensor 103 may be separately provided from the driving system of the first gas blowing unit 110. In addition, although a mode in which the chest compression unit 120 is driven by gas is indicated, the present invention is not limited thereto. The chest compression unit 120 may be, for example, driven by electricity.

In the cardiopulmonary resuscitation device 100 according to the embodiment, the gas used in the chest compression unit 120 is preferably reused in the first gas blowing unit 110. An amount of gas used can be saved. In addition, a gas cylinder can be reduced in size. When the gas used in the chest compression unit 120 is reused in the first gas blowing unit 110, the driving gas of the chest compression unit 120 is preferably pure oxygen. By blowing pure oxygen into the patient at recoiling, blood circulation is maintained and more efficient oxygenation can be performed without increasing intrathoracic pressure. Since it is possible to blow about five times as much oxygen as that in a case of blowing air, it is effective to prevent necrosis of the brain and the organs to improve cerebral performance category (CPC) and overall performance category (OPC).

The gas used in the chest compression unit 120 is preferably supplied to the first gas blowing unit 110 after the pressure thereof has been adjusted by a compressor to pressure suitable for blowing into the patient. The compressor is, for example, an oilless compressor. When disposing the compressor, it is preferable to dispose an air tank downstream the compressor in a gas passage between the discharge port of the cylinder 123 of the chest compression unit 120 and the hose outlet 112 of the first gas blowing unit 110. It is possible to suppress pulsation of pressure of air discharged from the compressor.

The first control unit 130 (130a, 130b) is, for example, a printed substrate. Although a main substrate 130a and a sub-substrate 130b are provided in FIG. 1 as an example of the first control unit 130, the present invention is not limited thereto. The first control unit 130 controls the first gas blowing unit 110 and the chest compression unit 120. In addition, the first control unit 130 can be externally controlled based on an external signal.

An external signal input unit 140 is, for example, a cable connecting terminal (not illustrated) or a receiving unit for a wireless signal or the like. An external signal input from the external signal input unit 140 is sent to the first control unit 130.

The cardiopulmonary resuscitation device 100 preferably has a mode-switching button (not illustrated). The mode-switching button (not illustrated) is, for example, a button provided in the housing 101 of the cardiopulmonary resuscitation device 100, an icon displayed on a touch panel (not illustrated) of the cardiopulmonary resuscitation device 100. In addition, the mode-switching button (not illustrated) may have a configuration in which when a connection cable as a signal transmission unit (not illustrated) is inserted into the connecting terminal as the external signal input unit 140, the mode-switching button is pushed by the insertion.

(Artificial Respirator)

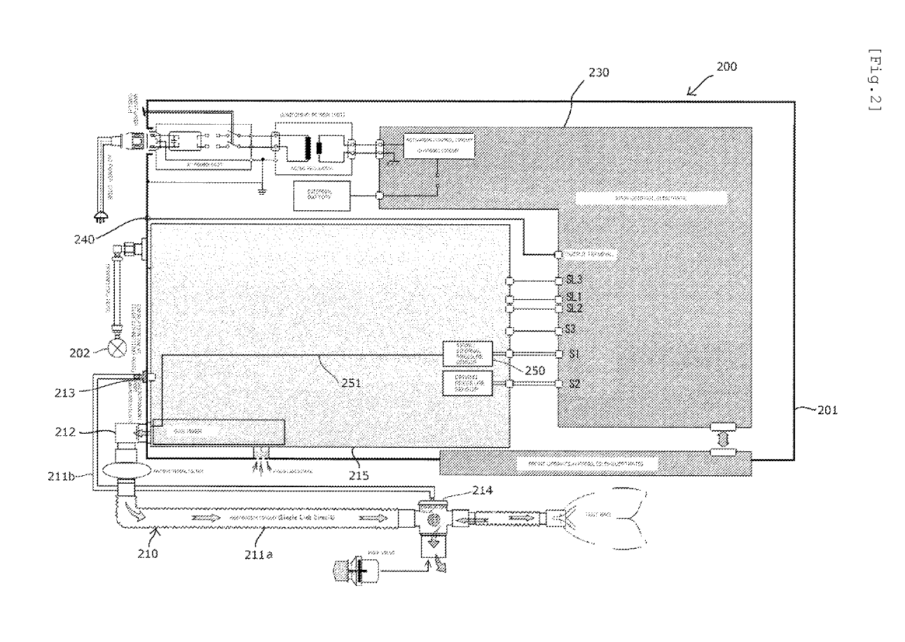

FIG. 2 is an example of a block diagram of an artificial respirator according to the embodiment. The artificial respirator 200 will be described with reference to FIG. 2. The artificial respirator 200 according to the embodiment has a second gas blowing unit 210 which blows breathable gas into a patient, a second control unit 230 which controls the second gas blowing unit 210 and generates an external signal including a remote control signal to the cardiopulmonary resuscitation device, an external signal output unit 240 which outputs the external signal generated by the second control unit 230 to the outside, and an airway internal pressure sensor 250 which detects pressure in the airway of the patient.

The artificial respirator 200 has a housing 201 which accommodates the second control unit 230, a driving system of the second gas blowing unit 210, and the like.

The second gas blowing unit 210 has an inhalation hose 211a for blowing breathable gas into the patient. The breathable gas is, for example, pure oxygen, oxygen-enriched air, or air. The breathable gas is more preferably pure oxygen. One end portion of the inhalation hose 211a is connected to a hose outlet 212 provided in the housing 201 of the artificial respirator 200. Another end portion of the inhalation hose 211a is coupled to a mask (not illustrated) to be attached to the patient or a tube for tracheal intubation (not illustrated). The second gas blowing unit 210 preferably has an exhalation hose 211b in addition to the inhalation hose 211a. Carbon dioxide in exhalation of the patient can be efficiently excluded from the system. One end portion of the exhalation hose 211b is connected to an exhalation valve 213. Another end portion of the exhalation hose 211b is coupled to an exhalation valve 214 disposed at a tip end portion of the inhalation hose 211a.

A driving system 215 of the second gas blowing unit 210 has the same basic configuration as that of the driving system of the first gas blowing unit 110. Here, regarding the driving system 215, a description of a configuration in common to that of the driving system of the first gas blowing unit 110 will be omitted, and a configuration different therefrom will be described. A driving gas supply source 202 of the second gas blowing unit 210 may be a portable gas cylinder or a stationary gas cylinder provided in an ambulance or a hospital. In the driving system 215 of the second gas blowing unit 210, a pressure reducer for ventilation 113 may be omitted and pressure of driving gas supplied from the driving gas supply source 202 may be reduced by a regulator (not illustrated) to gas pressure suitable for breathing. In addition, although the electromagnetic valve for ventilation 114 is used in the first gas blowing unit 110, it is preferable to use a valve capable of controlling a flow rate such as a flow rate adjusting valve (not illustrated) in the second gas blowing unit 210. Although the inhalation time of the breathable gas blown by the first gas blowing unit 110 can be adjusted only in a stepwise manner, inhalation time of the breathable gas blown by the second gas blowing unit 210 can be adjusted successively, for example, in a range of from 0.3 to 3.0 seconds. Furthermore, a ventilation amount of the breathable gas blown by the second gas blowing unit 210 can be adjusted in a range wider than that of the ventilation amount of the breathable gas blown by the first gas blowing unit 110, and for example, the amount is adjusted to 50 to 3000 ml for one time. Therefore, the driving system 215 of the second gas blowing unit 210 can adjust the flow rates more finely than the driving system of the first gas blowing unit 110. The flow rate adjusting valve (not illustrated) is controlled by the second control unit 230.

The second control unit 230 is, for example, a printed substrate. The second control unit 230 controls the driving system 215 of the second gas blowing unit 210. In addition, the second control unit 230 generates an external signal.

The external signal output unit 240 is, for example, a cable connecting terminal (not illustrated) or a transmitting unit for a wireless signal or the like, and outputs an external signal sent from the second control unit 230.

The airway internal pressure sensor 250 is a sensor capable of detecting from negative pressure to positive pressure, and detects pressure in the airway of the patient and outputs a pressure signal to the second control unit 230. The airway internal pressure sensor 250 detects, for example, pressure in a pipe 251 connected to the hose outlet 212 or the inhalation hose 211a, and regards the pressure in the pipe 251 as the pressure in the airway of the patient.

(Cardiopulmonary Resuscitation System)

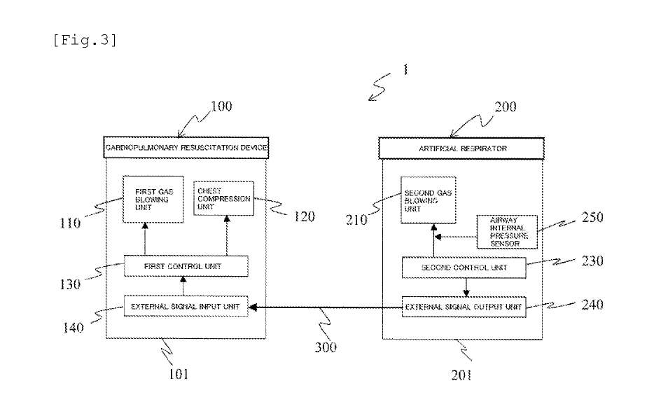

FIG. 3 is an example of a conceptual diagram of a cardiopulmonary resuscitation system according to the embodiment. The cardiopulmonary resuscitation system 1 according to the embodiment is equipped with the cardiopulmonary resuscitation device 100, the artificial respirator 200, and a signal transmission unit 300 which transmits an external signal to the external signal input unit 140 from the external signal output unit 240; and has a local mode in which the first gas blowing unit 110 and the chest compression unit 120 are operable and the second gas blowing unit 210 is stopped, and a remote mode in which the chest compression unit 120 and the second gas blowing unit 210 are operable and the first gas blowing unit 110 is stopped, and in the remote mode, the cardiopulmonary resuscitation device 100 and the artificial respirator 200 are capable of transmitting the external signal by the signal transmission unit 300, and the second control unit 230 controls the chest compression unit 120.

The cardiopulmonary resuscitation device 100 is, for example, a cardiopulmonary resuscitation device illustrated in FIG. 1.

The artificial respirator 200 is, for example, an artificial respirator illustrated in FIG. 2.

The signal transmission unit 300 is, for example, a connection cable or wireless communication.

The local mode is a mode in which chest compression and artificial respiration are performed only by the cardiopulmonary resuscitation device 100. Since there is the local mode, cardiopulmonary resuscitation can be started promptly at the emergency medical care field. In the local mode, the first control unit 130 controls the first gas blowing unit 110 and the chest compression unit 120. In the local mode, the cardiopulmonary resuscitation device 100 preferably has a synchronous mode in which the chest compression unit 120 repeats a predetermined number of chest compressions and waiting during which the chest compression unit 120 is temporarily stopped after the predetermined number of chest compressions, and when the chest compression unit 120 is in awaiting state, the first gas blowing unit 110 performs blowing of the breathable gas a predetermined number of times. Although a ratio of the chest compression and the artificial respiration in the synchronous mode is not particularly limited, for example, it is recommended in the guidelines that the chest compression and the artificial respiration are performed at a ratio of 30:2. In the synchronous mode in the local mode, the first control unit 130 controls the first gas blowing unit 110 to stop the blowing of the breathable gas while the chest compression unit 120 performs the chest compression. As a result, “passive ventilation” occurs every time the chest recoils after one performance of the chest compression. In addition, the first control unit 130 controls the first gas blowing unit 110 to perform the blowing of the breathable gas when the chest compression unit 120 is in a waiting state. As a result, “pushing” is performed. The control to cause the first gas blowing unit 110 to stop the blowing of the breathable gas is, for example, control to close the electromagnetic valve for ventilation 114 illustrated in FIG. 1. The control to cause the first gas blowing unit 110 to perform the blowing of the breathable gas is, for example, control to open the electromagnetic valve for ventilation 114 illustrated in FIG. 1. In addition, the local mode may have an asynchronous mode in which while the chest compression unit 120 successively performs the chest compression, the first gas blowing unit 110 performs the blowing of the breathable gas a predetermined number of times at predetermined time intervals. Although there is no particular limitation, the interval of the blowing of the breathable gas in the asynchronous mode is, for example, once per six seconds. In the asynchronous mode in the local mode, the first control unit 130 controls the first gas blowing unit 110 to perform the blowing of the breathable gas at predetermined timing such as once per six seconds. As a result, “pushing” is performed. In addition, the first control unit 130 controls the first gas blowing unit 110 to stop the blowing of the breathable gas when it is not the predetermined timing. As a result, “passive ventilation” occurs every time the chest recoils after one performance of the chest compression. Switching between the synchronous mode and the asynchronous mode in the local mode is performed, for example, by an adjusting knob (not illustrated) or a touch panel (not illustrated) provided on the housing 101 of the cardiopulmonary resuscitation device 100. In addition, an operator sets a ratio of the chest compression and the artificial respiration in the synchronous mode or a blowing interval of the breathable gas in the asynchronous mode, for example, through the adjusting knob (not illustrated) or the touch panel (not illustrated) provided on the housing 101 of the cardiopulmonary resuscitation device 100. In the local mode, the “passive ventilation” occurs every time the chest recoils after one performance of the chest compression in either of the synchronous mode or the asynchronous mode.

The remote mode is a mode in which the cardiopulmonary resuscitation device 100 performs chest compression only, and the artificial respirator 200 performs artificial respiration. At that time, an external signal can be transmitted to the cardiopulmonary resuscitation device 100 from the artificial respirator 200 by the signal transmission unit 300. In the remote mode, the second control unit 230 controls the second gas blowing unit 210 and the chest compression unit 120. The remote mode preferably has a synchronous mode in which the chest compression unit 120 repeats a predetermined number of chest compressions and waiting during which the chest compression unit 120 is temporarily stopped after the predetermined number of chest compressions, and the second gas blowing unit 210 blows the breathable gas into a patient at timing when the airway internal pressure sensor 250 detects negative pressure. In the synchronous mode in the remote mode, while the chest compression unit 120 performs the chest compression, the second control unit 230 controls the second gas blowing unit 210 to perform the blowing of the breathable gas when receiving a pressure signal indicating negative pressure from the airway internal pressure sensor 250, and controls the second gas blowing unit 210 to stop the blowing of the breathable gas when receiving a pressure signal indicating positive pressure or zero from the airway internal pressure sensor 250. As a result, “active ventilation” occurs every time the chest recoils after one performance of the chest compression. In addition, the second control unit 230 controls the second gas blowing unit 210 to perform the blowing of the breathable gas when the chest compression unit 120 is in a waiting state. The second control unit 230 performs “pushing”, for example, when counting the number of compressions performed by the chest compression unit 120 and the number of compressions reaches a predetermined number, or when detecting that the chest compression unit 120 has finished performing the predetermined number of chest compressions. As a result, “pushing” is performed. The control to cause the second gas blowing unit 210 to perform the blowing of the breathable gas is, for example, control to open the flow rate adjusting valve (not illustrated). The control to cause the second gas blowing unit 210 to stop the blowing of the breathable gas is, for example, control to close the flow rate adjusting valve (not illustrated). In addition, the remote mode may have an asynchronous mode in which the chest compression unit 120 successively performs the chest compression, and the second gas blowing unit 210 blows the breathable gas into the patient at timing when the airway internal pressure sensor 250 detects negative pressure. In the asynchronous mode in the remote mode, while the chest compression unit 120 performs the chest compression, the second control unit 230 controls the second gas blowing unit 210 to perform the blowing of the breathable gas when receiving a pressure signal indicating negative pressure from the airway internal pressure sensor 250, and controls the second gas blowing unit 210 to stop the blowing of the breathable gas when receiving a pressure signal indicating positive pressure or zero from the airway internal pressure sensor 250. As a result, “active ventilation” occurs every time the chest recoils after one performance of the chest compression. In addition, the second control unit 230 controls the first gas blowing unit 110 to perform the blowing of the breathable gas at predetermined timing such as once in six seconds. As a result, “pushing” is performed. A method for performing switching between the synchronous mode and the asynchronous mode in the remote mode is the same as that in the local mode. Setting of a ratio of the chest compression and the artificial respiration in the synchronous mode or a blowing interval of the breathable gas in the asynchronous mode is also the same as that in the local mode. What is meant by that the airway internal pressure sensor 250 detects negative pressure is that the intrathoracic pressure has decreased at recoiling of the chest wall of the patient pushed by the chest compression. In the remote mode, the “active ventilation” occurs every time the chest recoils after one performance of the chest compression in either of the synchronous mode or the asynchronous mode. Therefore, it is possible to further increase a cardiopulmonary resuscitation rate. The active ventilation is more preferably ventilation by pure oxygen with the use of pure oxygen as the breathable gas.

The second control unit 230 generates, for example, an external signal including a remote control signal instructing the chest compression unit 120 to execute the chest compression. The external signal including a remote control signal is output from the external signal output unit 240 and input to the external signal input unit 140 by the signal transmission unit 300. The external signal including a remote control signal input by the external signal input unit 140 is sent to the first control unit 130. When inputting the remote control signal, the first control unit 130 drives the chest compression unit 120. In the manner as described above, the second control unit 230 remote-controls the chest compression unit 120. In addition, when the pressure detected by the airway internal pressure sensor 250 is negative pressure, the second control unit 230 outputs, to the flow rate adjusting valve (not illustrated), a signal which causes the flow rate adjusting valve (not illustrated) to open.

In the cardiopulmonary resuscitation system 1 according to the embodiment, preferably, the cardiopulmonary resuscitation device 100 has a mode-switching button (not illustrated) for performing switching between the local mode and the remote mode, or the external signal includes a mode-switching signal for performing switching between the local mode and the remote mode. Switching from the local mode to the remote mode can be performed easily and quickly.

The mode-switching signal is, for example, an electric signal generated when a connection cable is inserted into a connecting terminal as the external signal input unit 140, or an external signal generated by the second control unit 230. In addition, the remote control signal may also serve as the mode-switching signal.

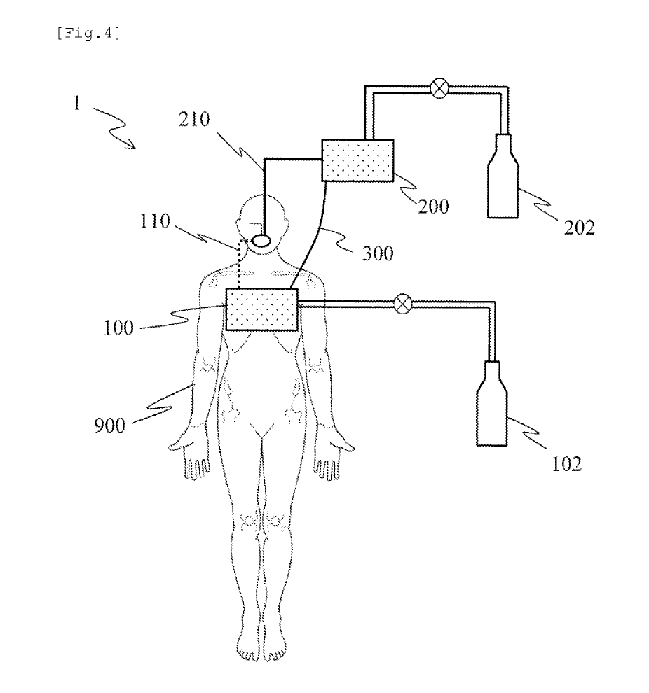

FIG. 4 is a drawing for explaining use of the cardiopulmonary resuscitation system according to the embodiment. An example of the use of the cardiopulmonary resuscitation system in the emergency medical care field will be described with reference to FIG. 4. First, the cardiopulmonary resuscitation device 100 is mounted on a patient 900, and cardiopulmonary resuscitation is performed in the local mode. In other words, the chest compression unit (not illustrated) compresses the chest of the patient 900, and the first gas blowing unit 110 blows the breathable gas into the patient 900. Subsequently, the local mode is switched to the remote mode at appropriate timing, and the cardiopulmonary resuscitation is performed in the remote mode. In other words, the chest compression unit (not illustrated) compresses the sternum of the patient 900, and the second gas blowing unit 210 blows the breathable gas into the patient 900. In the manner as described above, the cardiopulmonary resuscitation device according to the embodiment can start cardiopulmonary resuscitation promptly in the local mode in an initial stage of the emergency medical care, and can perform the cardiopulmonary resuscitation with higher accuracy by switching the local mode to the remote mode. As a result, a cardiopulmonary resuscitation rate is expected to increase. Although in FIG. 4, a mode is illustrated in which the connection cable as the signal transmission unit 300 connects the cardiopulmonary resuscitation device 100 and the artificial respirator 200, in the local mode, the connection cable may be connected only to the cardiopulmonary resuscitation device 100, may be connected only to the artificial respirator 200, or may be connected to neither the cardiopulmonary resuscitation device 100 nor the artificial respirator 200. In the local mode, when the connection cable is connected only to the cardiopulmonary resuscitation device 100, is connected only to the artificial respirator 200, or is connected to neither the cardiopulmonary resuscitation device 100 nor the artificial respirator 200, the cardiopulmonary resuscitation device 100 and the artificial respirator 200 are connected by the connection cable at timing when the local mode is switched to the remote mode.

When the local mode is switched to the remote mode, the first gas blowing unit 110 is switched to the second gas blowing unit 210 simultaneously. Although the switching from the first gas blowing unit 110 to the second gas blowing unit 210 is not particularly limited, the switching is performed, for example, by detaching a mask of the first gas blowing unit 110 from the patient 900 and instead thereof, coupling a mask of the second gas blowing unit 210 to the patient 900, by detaching a hose of the first gas blowing unit 110 from the tube for tracheal intubation and instead thereof, coupling a hose of the second gas blowing unit 210 to the tube for tracheal intubation, or by detaching a hose of the first gas blowing unit 110 from the hose outlet of the cardiopulmonary resuscitation device 100 and instead thereof, inserting the hose into the hose outlet of the artificial respirator 200.

Preferably, the cardiopulmonary resuscitation system 1 according to the embodiment is further equipped with a monitor for measuring regional saturation of oxygen (not illustrated). The monitor for measuring regional saturation of oxygen monitors regional saturation of oxygen (rSO₂) of a patient by using a near infrared ray. The monitor for measuring regional saturation of oxygen is preferably mounted to the patient 900 at an initial stage of the emergency medical care such as before the cardiopulmonary resuscitation device 100 is mounted to the patient 900 or immediately after the cardiopulmonary resuscitation device 100 is mounted thereto. It is possible for doctors or emergency life-saving technicians to more appropriately perform, in the local mode, setting of a ratio of the chest compression and the artificial respiration in the synchronous mode or a blowing interval of the breathable gas in the asynchronous mode, and switching from the local mode to the remote mode, and in the remote mode, setting of a ratio of the chest compression and the artificial respiration in the synchronous mode or a blowing interval of the breathable gas in the asynchronous mode in accordance with the regional saturation of oxygen monitored by the monitor for measuring regional saturation of oxygen. As a result, it is possible to significantly contribute to an increase in an emergency life-saving rate of patients, an increase in a cardiopulmonary resuscitation rate of patients, and an increase in a social rehabilitation rate of patients after saving their lives.

### REFERENCE SIGNS LIST

1 Cardiopulmonary resuscitation system
10 Arch portion
11 Top surface portion
12 Right and left side surface portions
13 Fixing unit
14 Display unit
20 Vertical rod
21 Scale
30 Back plate
100 Cardiopulmonary resuscitation device
101 Housing of cardiopulmonary resuscitation device
102 Driving gas supply source of first gas blowing unit
103 Driving gas pressure sensor
110 First gas blowing unit
111 Hose
112 Hose outlet
113 Pressure reducer for ventilation
114 Electromagnetic valve for ventilation
115 Positive pressure safety valve
116 Airway internal pressure sensor
120 Chest compression unit
121 Impact hammer
121a Impact hammer rod
121b Impact head pad
122 Elevating unit
122 Elevating mechanism
123 Cylinder
124 Piston
125 Spring
126 Compression depth adjuster
127 Electromagnetic valve for compression
130 First control unit
130a Main substrate
130b Sub-substrate
140 External signal input unit
151 to 159 Pipe
200 Artificial respirator
201 Housing of artificial respirator
202 Driving gas supply source of second gas blowing unit
210 Second gas blowing unit
211a Inhalation hose
211b Exhalation hose
212 Hose outlet
213 Exhalation valve
214 Exhalation valve
215 Driving system of second gas blowing unit
230 Second control unit
240 External signal output unit
250 Airway internal pressure sensor
251 Pipe
300 Signal transmission unit
900 Patient

1. A cardiopulmonary resuscitation system comprising:
a cardiopulmonary resuscitation device that has a first gas blowing unit that blows breathable gas into a patient, a chest compression unit that compresses the chest of the patient, a first control unit that controls the first gas blowing unit and the chest compression unit, and an external signal input unit that inputs an external signal including a remote control signal instructing the chest compression unit to execute the chest compression;
an artificial respirator that has a second gas blowing unit that blows breathable gas into the patient, a second control unit that controls the second gas blowing unit and generates the external signal, an external signal output unit that outputs the external signal generated by the second control unit to outside, and an airway internal pressure sensor that detects pressure in the airway of the patient; and
a signal transmission unit that transmits the external signal to the external signal input unit from the external signal output unit, and having:
a local mode in which the first gas blowing unit and the chest compression unit are operable and the second gas blowing unit is stopped; and
a remote mode in which the chest compression unit and the second gas blowing unit are operable and the first gas blowing unit is stopped, wherein
in the remote mode, the cardiopulmonary resuscitation device and the artificial respirator are capable of transmitting the external signal by the signal transmission unit, and the second control unit controls the chest compression unit.

2. The cardiopulmonary resuscitation system according to claim 1, wherein the cardiopulmonary resuscitation device has a mode-switching button for performing switching between the local mode and the remote mode, or
the external signal includes a mode-switching signal for performing switching between the local mode and the remote mode.

3. The cardiopulmonary resuscitation system according to claim 1, wherein in the remote mode, the second gas blowing unit executes blowing of the breathable gas at timing when the airway internal pressure sensor detects negative pressure.

4. A cardiopulmonary resuscitation device comprising:
a gas blowing unit that blows breathable gas into a patient;
a chest compression unit that compresses the chest of the patient;
a control unit that controls the gas blowing unit and the chest compression unit; and
an external signal input unit that inputs an external signal including a remote control signal instructing the chest compression unit to execute the chest compression.

5. An artificial respirator comprising:
a gas blowing unit that blows breathable gas into a patient;
a control unit that controls the second gas blowing unit and generates an external signal including a remote control signal to a cardiopulmonary resuscitation device;
an external signal output unit that outputs the external signal generated by the control unit to outside; and
an airway internal pressure sensor that detects pressure in the airway of the patient.

6. The cardiopulmonary resuscitation system according to claim 2, wherein in the remote mode, the second gas blowing unit executes blowing of the breathable gas at timing when the airway internal pressure sensor detects negative pressure.
